(19) United States
(12) Patent Application Publication
(10) Pub. No.: US 2018/0064394 A1
(43) Pub. Date: Mar. 8, 2018

(54) VIRTUAL PROTOTYPING INTEGRATED ELECTRONICS IN APPAREL USING PHYSIOLOGIC-ENABLED AVATAR

(71) Applicant: Intel Coporation, Santa Clara, CA (US)

(72) Inventor: Eric Lewallen, Portland, OR (US)

(21) Appl. No.: 15/259,134

(22) Filed: Sep. 8, 2016

(51) Int. Cl. A61B 5/00; A41D 1/00; A41H 43/00; G06F 17/50

(52) U.S. Cl. CPC A61B 5/6804; A41D 1/005; G01N 27/041; G06F 17/50; A41H 43/00

(57) ABSTRACT

Systems, apparatuses and methods incorporate biometric testing and standards, textile standards, processor specifications and conductive fabric specifications to provide a way to efficiently design and produce technology embedded garments and/or apparel. The systems, apparatuses and methods may provide a design visualizer to retrieve specifications for conductive materials, decorative materials, sensors, design patterns and physiological models to design and produce technology embedded garments and/or apparel to monitor one or more biosignals. Using the design visualizer, the design patterns may be edited and/or refined to position the sensors to increase (e.g. maximize) performance of the sensors and/or accuracy of the sensors measurements and biosignals measurements, and reduce (e.g., minimize) the number of sensors. Additionally, the design visualizer may provide a visual heat map and overlay of positions and zones that identify recommended positions to locate the sensors based on one or more physiological models.

### TECHNICAL FIELD

Embodiments generally relate to monitoring the physiology of biological systems. More particularly, embodiments relate to designing and producing technology embedded garments and apparel to monitor the physiology of biological systems.

### BACKGROUND

Current three dimensional (3D) simulation tools enable garment and apparel designers to assess the design, color, and fabric drape of a garment and/or apparel via simulation. These 3D tools fail to provide a way to explore viable positions to locate sensors and to make informed integration decisions based on physiological modeling. Moreover, prototyping and testing of physical garment samples is time consuming and expensive.

### BRIEF DESCRIPTION OF THE DRAWINGS

The various advantages of the embodiments will become apparent to one skilled in the art by reading the following specification and appended claims, and by referencing the following drawings, in which:

FIG. 1 is an illustration of an example of a technology embedded garment system configuration according to an embodiment;

FIG. 2 is a block diagram of an example of technology embedded garment system according to an embodiment;

FIG. 3 is a flowchart of an example of a method of generating a design pattern of a technology embedded garment according to an embodiment;

FIG. 4A is an illustration of an example of a technology embedded garment with conductive pathways according to an embodiment;

FIG. 4B is an illustration of an example of a heat map of recommended sensor areas according to an embodiment;

FIG. 5A is an illustration of an example of a design visualizer display area according to an embodiment;

FIG. 5B is an illustration of an example of a design visualizer display area according to another embodiment;

FIG. 6 is a block diagram of an example of a processor according to an embodiment; and

FIG. 7 is a block diagram of an example of a computing system according to an embodiment.

### DESCRIPTION OF EMBODIMENTS

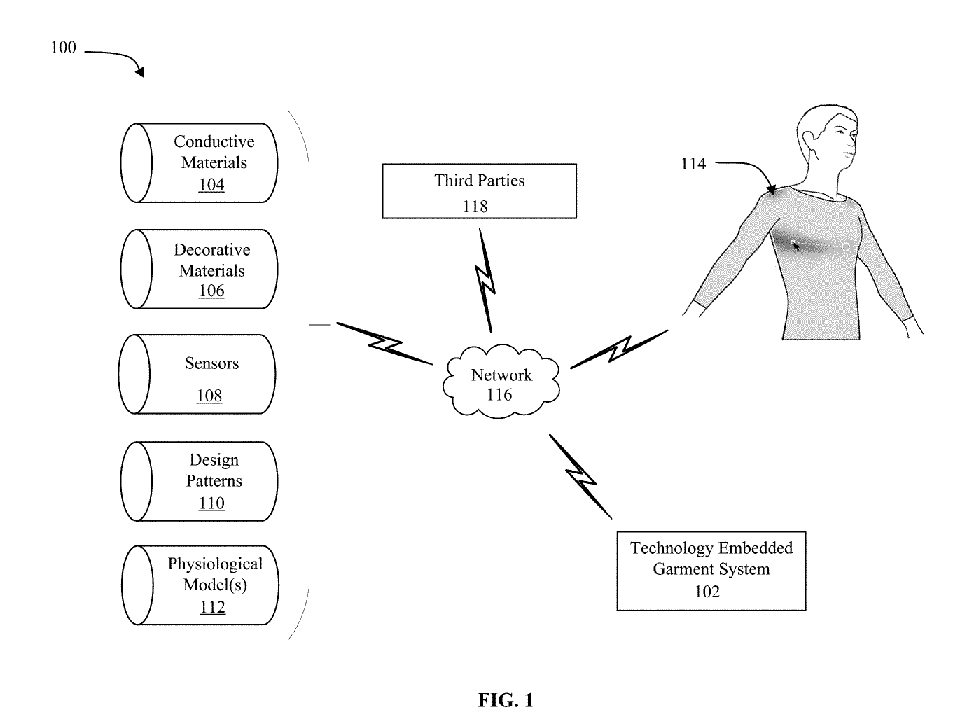

Turning now to FIG. 1, an example is illustrated of a technology embedded garment (TEG) system configuration 100. The TEG system configuration 100 may include a TEG system 102, which may include, receive and/or retrieve specifications for conductive materials 104, decorative materials 106, sensors 108, design patterns 110, and physiological models 112 for one or more wearers of technology embedded garments. The conductive materials 104 may include materials that conduct electrical and/or thermal signals including conductive fabric, conductive fibers, conductive inks and coatings, wires, wire mesh, woven metals and composite materials. The decorative materials 106 may include fabrics, plastics, leather and various other materials usable for garments and apparel.

The TEG system 102 may be used (e.g., by a clothing designer, manufacturer, etc.) to design technology embedded garments and/or apparel embedded with sensors to measure one or more biosignals. For biosignals, the sensors 108 may measure a change in electric current produced by a sum of an electrical potential difference across biological tissue, organs, cell systems and nervous systems. The sensors 108 may measure a change in electric resistance produced by the conductive materials 104 designed by modifying the conductive materials 104 in simulation to compare electrical properties between different simulated conductive materials 104 including one or more of conductive threads, fabrics, inks or composites. The sensors 108 may also measure one or more thermal signals and/or temperature differences across mechanical devices, components and systems, and/or biological systems including tissue, organs, cell systems and nervous systems. More particularly, the TEG system 102 may generate the design of and produce a technology embedded garment 114. The TEG system 102 may communicate with various components of the TEG system configuration 100 via a network 116 (e.g., the Internet). The TEG system 102 may provide a way to reduce (e.g., minimize) the amount of conductive materials, decorative materials and number of sensors used to increase (e.g. maximize) accuracy of sensor measurements, while reducing the production time, resources and/or costs to produce the technology embedded garment 114.

In one embodiment, the TEG system 102 may monitor the sensors embedded in the technology embedded garment 114 and/or apparel produced by the TEG system 102, while the wearer is wearing the technology embedded garment 114 and/or apparel, and provide sensor data to the wearer and/or one or more third parties 118 (e.g., doctor, physical therapist, employer, service providers) for use to monitor the condition of the wearer, and/or to refine (e.g., iterate) the design patterns 110 and physiological models 112. The third parties 118 may provide the conductive materials 104, decorative materials 106, sensors 108, design patterns 110 and physiological models 112, and/or provide recommendations (e.g., nutritional information, medications, activities and/or other technology embedded garments and apparel) to the wearer based on the sensor data.

In another embodiment, the TEG system 102 may also monitor the sensors embedded in the technology embedded garment 114 produced by the TEG system 102 to generate and/or refine the physiological model 112 of the wearer and/or intended wearer of the technology embedded garment 114 and/or apparel.

The TEG system 102 enables garment and apparel designers to use 3D modeling to visualize and assess the performance of integrated electrical components in smart garments. With the rise of smart fabrics and electronically integrated apparel the TEG system 102 enables designers and engineers to make similarly informed decisions about electronics integration prior to physical prototyping and testing. The TEG system 102 employs 3D modeling to provide virtual integration of electrical components in garments, which enables designers to assess the viability of conductive pathways and how integrated electronics may affect fabric drape and hand of the finished garment. The TEG system 102 simulates the conductive pathways, simulates resistance measurements based on selected materials and flags (e.g., presents to the user for resolution) potential integration errors.

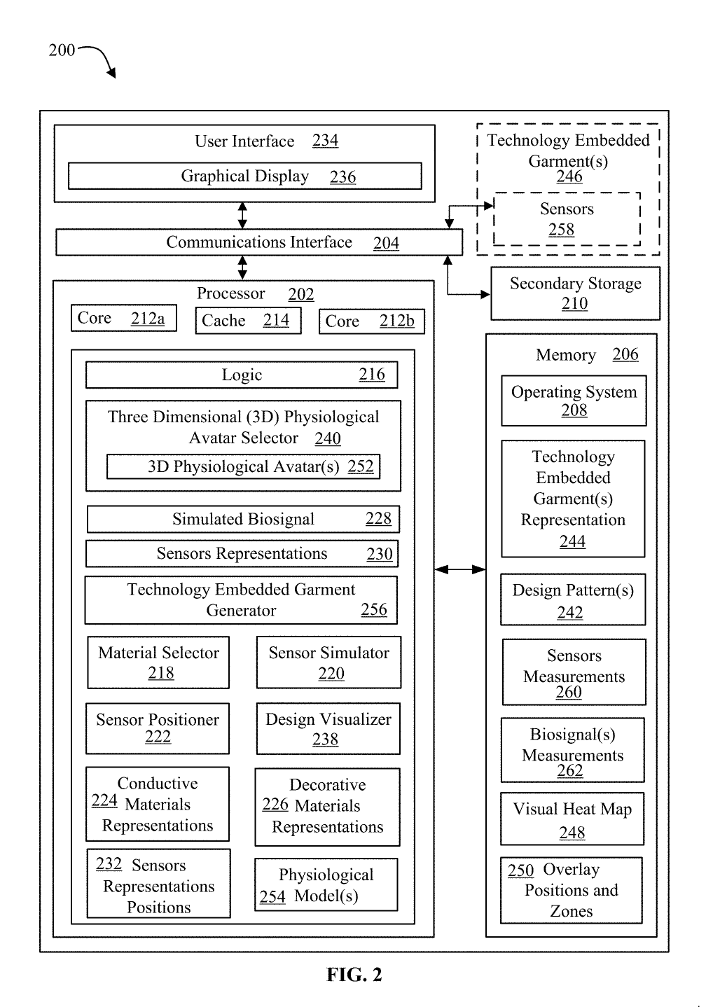

Turning now to FIG. 2, a block diagram is illustrated of an example of a technology embedded garment (TEG) system 200. The TEG system 200 which may be readily substituted for the system 102 (FIG. 1), already discussed, may include a processor 202, a communications interface 204 and memory 206 coupled to the processor 202. The processor 202 runs an operating system (OS) 208. The memory 206 may be external to the processor 202 (e.g., external memory), and/or may be coupled to the processor 202 by, for example, a memory bus. In addition, the memory 206 may be implemented as main memory. The memory 206 may include, for example, volatile memory, non-volatile memory, and so on, or combinations thereof. For example, the memory 206 may include dynamic random access memory (DRAM) configured as one or more memory modules such as, for example, dual inline memory modules (DIMMs), small outline DIMMs (SODIMMs), etc., read-only memory (ROM) (e.g., programmable read-only memory (PROM), erasable PROM (EPROM), electrically EPROM (EEPROM), etc.), phase change memory (PCM), and so on, or combinations thereof. The memory 206 may include an array of memory cells arranged in rows and columns, partitioned into independently addressable storage locations. The processor 202 and/or operating system 208 may use a secondary memory storage 210 with the memory 206 to improve performance, capacity and flexibility of the TEG system 200.

The TEG system 200 may include cores 212a, 212b that may execute one or more instructions such as a read instruction, a write instruction, an erase instruction, a move instruction, an arithmetic instruction, a control instruction, and so on, or combinations thereof. The cores 212a, 212b may, for example, execute one or more instructions to move data (e.g., program data, operation code, operand, etc.) between a cache 214 or a register (not shown) and the memory 206 and/or the secondary memory storage 210, to read the data from the memory 206, to write the data to the memory 206, to perform an arithmetic operation using the data (e.g., add, subtract, bitwise operation, compare, etc.), to perform a control operation associated with the data (e.g., branch, etc.), and so on, or combinations thereof. The instructions may include any code representation such as, for example, binary code, octal code, and/or hexadecimal code (e.g., machine language), symbolic code (e.g., assembly language), decimal code, alphanumeric code, higher-level programming language code, and so on, or combinations thereof. Thus, for example, hexadecimal code may be used to represent an operation code (e.g., opcode) of an x86 instruction set including a byte value “00” for an add operation, a byte value “8B” for a move operation, a byte value “FF” for an increment/decrement operation, and so on.

The TEG system 200 may include logic 216 to coordinate processing among various components and/or subsystems of the TEG system 200. The TEG system 200 may include a material selector 218, a sensor simulator 220 and a sensor positioner 222. The material selector 218 may provide for user selection representations of one or more conductive materials 224 that include conductive pathways, and representations of one or more decorative materials 226 for a representation of a technology embedded garment 244 and/or apparel.

The sensor simulator 220 may monitor one or more simulated biosignal 228 at one or more representations of sensors 230 positioned along the conductive pathways of the conductive materials 224. The sensor simulator 220 may monitor the simulated biosignal 228 by measuring a change in simulated electric current produced by a sum of an electrical potential difference across one or more of a simulated tissue, organ, cell system or nervous system. The sensor simulator 220 may measure a change in electric resistance produced by the conductive materials 224 modified in a simulation to compare electrical properties between different simulated conductive materials including one or more of conductive threads, fabrics, inks or composites. The simulated biosignal 228 may include one or more of simulated bioelectrical signals, electrical signals, non-electrical signals or time-varying signals. In one embodiment, the sensor simulator 220 may record the sensors measurements 260 and biosignal(s) measurements 262 of the sensors 258 and biosignal(s) 264.

The sensor positioner 222 may determine sensors representations positions 232 (e.g., locations), based on one or more physiological models 254 of one or more intended wearers of the technology embedded garment 246 and/or apparel, and previous sensors measurements and biosignal(s) measurements recorded by sensors embedded in previously worn technology embedded garments and/or apparel. The sensor positioner 222 may allow the user to position the representations of the sensors 230 to reduce (e.g., minimize) the number of representations of sensors and increase (e.g. maximize) accuracy of the sensors measurements 260 recorded by the sensors 258 of the biosignal(s) of the intended wearer. The sensors 258 may include multimodal and/or unimodal biosensors, biometric sensors, and/or equipment and device sensors. The sensors measurements 260 and biosignal(s) measurements 262 may be used by the TEG system 200 to train the sensor positioner 222 to improve recommendations for the positioning of the representations of the sensors 230. The sensors 230, 258 (e.g., simulated and embedded in the technology embedded garments and/or apparel) may measure physiological responses of the wearer, including, for example, electrocardiogram (ECG), Galvanic skin response (GSR), electromyogram (EMG), electroencephalogram (EEG), Mechanomyogram (MMG), electrooculography (EOG), magnetoencephalogram (MEG) and/or body temperature.

The TEG system 200 may also include a user interface 234 that includes a graphical display 236 to present the user a design visualizer 238 (e.g., CLO ATELIER configured with the technology embedded garment system logic 216 as a plug-in) and a three dimensional (3D) physiological avatar selector 240. In one embodiment, the design visualizer 238 may be implemented by configuring a 3D design tool (e.g., a CLO ATELIER) with a plug-in of the logic 216 of the technology embedded garment system 200. The design visualizer 238 may display (e.g., present) design patterns 242 for the user to view (e.g., visualize), select and/or edit designs of representations of technology embedded garments 244 and/or apparel, and position the sensors representations 230 for the sensors 258 to be embedded in the technology embedded garments 246 and/or apparel.

In one embodiment, the TEG system 200 may include, receive and/or retrieve the design patterns 242 that the TEG system 200 virtually assembles, and renders onto the 3D physiological avatar selector 240 (e.g., a 3D avatar model). The TEG system 200 may use the design visualizer 238 to apply a compute module and the conductive pathways to the representation of the design pattern of the technology embedded garment 246 and/or apparel. The TEG system 200 may retrieve and/or import the design patterns 242 from a computer aided design (CAD) program (e.g., software application) and/or system. The design visualizer 238 may include a property editor that the user may use to view properties of the technology embedded garment 244 and/or apparel. The properties of the technology embedded garment 244 and/or apparel may include processor type and battery type for the sensors 230, 258 and virtual resistance measurements of the conductive pathways based on the conductive materials 224 and the decorative materials 226 (e.g., textile specifications). The TEG system 200 may upload processor specifications (e.g., Intel® Curie™ or D1000) for the sensors 230, 258 into the representation (e.g., model) of the technology embedded garment 244 and/or apparel to generate power numbers for the technology embedded garment 244 and/or apparel, based on the proposed design of the technology embedded garment 244 and/or apparel. The user may modify the conductive pathways by length and width, and the representations of the conductive materials 224 and analyze resulting measurements until the technology embedded garment 244 and/or apparel is configured to satisfy user specified performance thresholds (e.g., reduce/minimize the number of sensors and/or amount of conductive materials).

The design visualizer 238 may display a visual heat map 248 and overlay 250 of positions and zones for the one or more representations of sensors 230 (e.g., sensors representations). The visual heat map 248 and overlay 250 may identify and/or indicate, using a color spectrum, recommended positions (e.g., locations, placement) for the representations of sensors 230 to increase (e.g. maximize) performance of the sensors 258 and/or accuracy of measurements to be recorded by the sensors 258 of the biosignal(s). The color spectrum may include the colors red, orange, yellow, green and blue to indicate best to least recommended positions for the sensors 258 to be embedded in the technology embedded garment 246 and/or apparel. The visual heat map 248 may be presented as an overlay 250 of positions and zones to a bio-accurate 3D physiological avatar 252.

The 3D physiological avatar selector 240 may provide (e.g., present and/or display) bio-accurate 3D physiological avatars 252 for selection based on one or more potential and/or intended wearers (e.g., gender, size, biological—gender, human, animal or plant, non-biological—equipment or device) of the technology embedded garment 246. The 3D physiological avatar selector 240 may generate the simulated biosignal 228 for a potential and/or intended wearer of the technology embedded garment 246.

A biosignal may include a signal produced by a biological system that may be measured and monitored. The biosignal simulated by the simulated biosignal 228 may include one or more of bioelectrical signals, electrical signals, non-electrical signals, time-varying signals or spatial parameter variations (e.g., the nucleotide sequence determining the genetic code). The non-electrical signals may include mechanical signals (e.g., mechanomyogram MMG), acoustic signals (e.g., phonetic and non-phonetic utterances, breathing), chemical signals (e.g., pH, oxygenation) and optical signals (e.g., movements).

The 3D physiological avatar 252 may be responsive to the representations of the embedded technology (e.g., sensors 258 and conductive materials) and the technology embedded garment 246. For example, the 3D physiological avatar 252 may be responsive to simulated movements and/or simulated activities in which a wearer may engage while wearing the technology embedded garment 246, applied pressure from an actuated garment, impact of electrical signals on the body such as transcutaneous electrical nerve stimulation (TENS) and muscle electrostimulation for sport training. The 3D physiological avatar 252 may be customized for one or more activities (e.g., sports, type of work, motion, operations), environments (e.g., temperature, elevation, aquatic, terrestrial, pressure, atmosphere) and one or more wearer profiles (e.g., size, biology—health, gender, human, animal and/or plant, non-biological characteristics—robots, equipment and/or devices).

The TEG system 200 may also include a technology embedded garment generator 256 to generate a design pattern 242 of and produce the technology embedded garment 246 and/or apparel embedded with sensors 258 represented by the representations of the sensors 230.

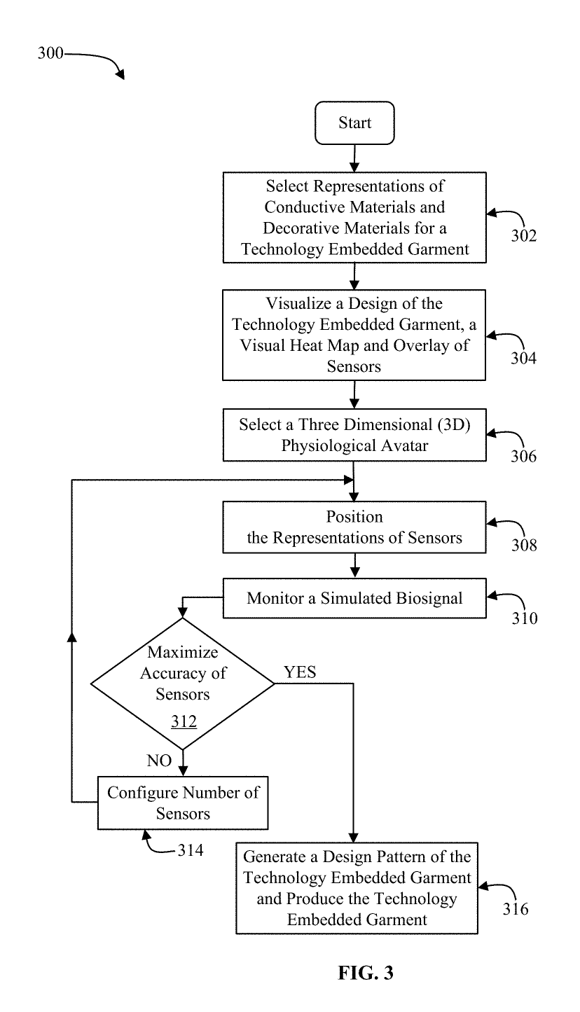

FIG. 3 shows a method 300 of generating a design pattern of the technology embedded garment. The method 300 may be implemented as a module or related component in a set of logic instructions stored in a non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof. For example, computer program code to carry out operations shown in the method 300 may be written in any combination of one or more programming languages, including an object oriented programming language such as JAVA, SMALLTALK, C++ or the like and conventional procedural programming languages, such as the “C” programming language or similar programming languages.

Illustrated processing block 302 provides for selecting representations of one or more conductive materials and one or more decorative materials for a technology embedded garment. The selection of conductive materials and decorative materials includes specifications of the conductive materials and decorative materials. The conductive materials include conductive pathways where one or more sensors (e.g., multimodal and/or unimodal biosensors, biometric sensors, and/or equipment and device sensors) may be positioned.

Illustrated processing block 304 may provide for visualizing a design (e.g., pattern), using a design visualizer, to edit the design of the representation of the technology embedded garment and position the representations of the sensors. The design visualizer may present the properties of the conductive materials, decorative materials and sensors to be used to produce a technology embedded garment and/or apparel.

Illustrated processing block 306 may provide for selecting a three dimensional (3D) physiological avatar from an avatar selector. The 3D physiological avatar selector may provide avatars for selection based on one or more potential wearers (e.g., gender, size, biological—gender, human, animal or plant, non-biological—equipment or device). The 3D physiological avatar selector may generate a simulated biosignal for the 3D physiological avatar for input to the embedded technology in the garment. The biosignal may include one or more of bioelectrical signals, electrical signals, non-electrical signals, time-varying signals or spatial parameter variations. The 3D physiological avatar may be responsive to the embedded technology and the garment (e.g., responsive to movements and/or activities in which a wearer may engage while wearing the embedded technology garment). The 3D physiological avatar may be customized for one or more activities, environments and one or more wearer profiles.

Illustrated processing block 308 may provide for positioning the representations of the sensors to reduce (e.g., minimize) the number of the representations of sensors and increase (e.g., maximize) accuracy of the representations of the sensors to measure the at least one simulated biosignal. The design visualizer may display a visual heat map and overlay that identifies positions and zones for the one or more representations of sensors to monitor the simulated biosignal.

Illustrated processing block 310 may provide for monitoring the simulated biosignal at one or more of the representations of the sensors positioned along the conductive pathways of the conductive materials. A determination may be made at processing block 312 as to whether the positioning of the representations of the sensors increases (e.g., maximizes) the accuracy of the representations of sensors to measure the simulated biosignal. If the accuracy of the representations of sensors to measure the simulated biosignal is not increased (e.g., maximized) based on the position and number of sensors, then processing block 314 may provide for editing (e.g., configuring) the number of sensors, and/or subsequently, as provided by processing block 312 the positioning (e.g., re-positioning) of the sensors. The TEG system determines sensor positions for various sensing modalities, and for the sensors to function within an acceptable range. The TEG system enables the designer to visualize and make informed decisions regarding accuracy of measurements over fit of the technology embedded garment based on accurate biometric models and modify the design of the technology embedded garment accordingly. Once the positioning and/or number of sensors is configured, illustrated processing block 316 may provide for generating a design pattern of the technology embedded garment and producing the technology embedded garment.

The TEG system may enable the user to design technology embedded garments and apparel using informed decisions and trade-offs (e.g., assessing fit, style and function). The TEG system may further integrate the sensor data recorded from the technology embedded garment and/or apparel (e.g., sensor data) with a product lifecycle management (PLM) tools to enable costing models to be generated on components bill of materials (BOM), and inform manufacturing instructions towards direct integration, for example robotic sewing and knitting machines.

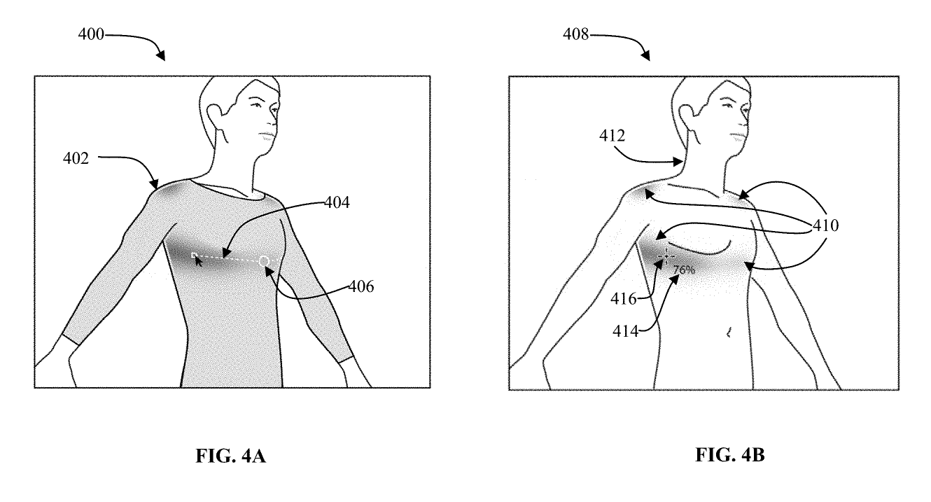

Turning now to FIG. 4A, an example 400 is illustrated of a technology embedded garment 402 with conductive pathways 404 according to an embodiment. One or more sensors 406 may be positioned along one or more conductive pathways 404 of the technology embedded garment 402 and/or apparel, where the number and position of the sensors and conductive materials may be configured to increase (e.g., maximize) the accuracy of the sensors measurements and the biosignal(s) measurements, while reducing (e.g., minimizing) the number of sensors and amount of conductive materials and/or decorative materials used to produce (e.g., generate) the technology embedded garment 402 and/or apparel.

Turning now to FIG. 4B, an example 408 is illustrated of a heat map of recommended sensor areas 410. The heat map of recommended sensor areas 410 may be presented as an overlay of positions and zones to a 3D physiological avatar 412. The heat map of recommended sensor areas 410 overlay of positions and zones to locate sensors to measure and/or record one or more biosignals. The heat map of recommended sensor areas 410 may include one or more confidence values 414 (e.g., 0-100%) that indicates a level of performance and/or accuracy of one or more sensors to record a biosignal, and/or integrity (e.g., strength) of the biosignal at a position 416 of the 3D physiological avatar 412.

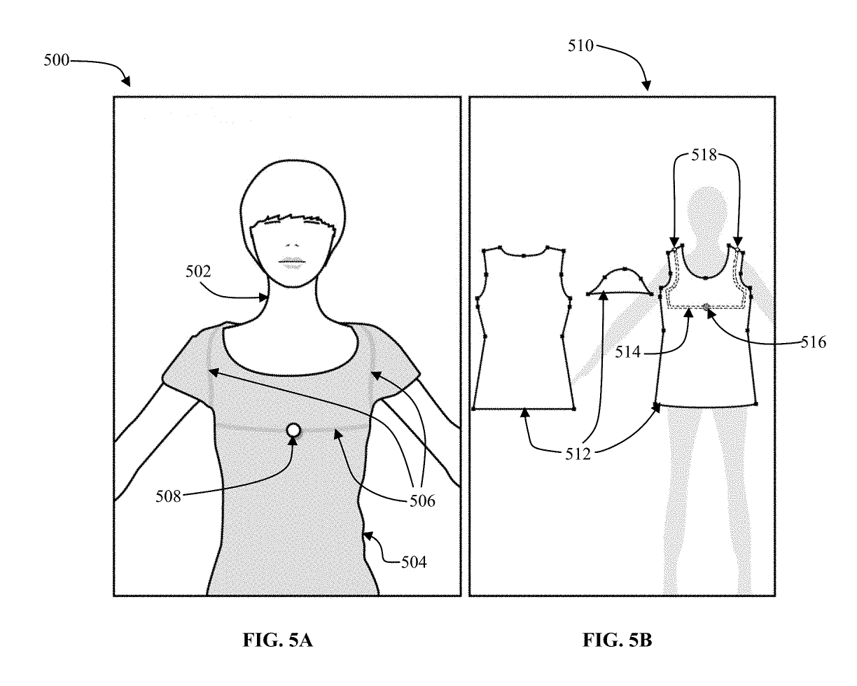

Turning now to FIG. 5A, an example is illustrated of a design visualizer display area 500 displaying a 3D physiological avatar 502 and a design pattern representation of a technology embedded garment with decorative materials 504 and conductive pathways 506 according to an embodiment. The conductive pathways 506 may include one or more sensors 508. The design visualizer area 500 provides the user a way to view and edit the design pattern representation of the technology embedded garment, including the decorative materials 504, the conductive pathways 506, and position and number of and position the representations of the one or more sensors 508.

Turning now to FIG. 5B, an example is illustrated of another design visualizer display area 510 displaying a design pattern representation of a technology embedded garment with decorative materials 512 and conductive pathways 514 according to another embodiment. The design visualizer display area 510 provides the user another way to view and edit the design pattern representation of the technology embedded garment, including the decorative materials 512, the conductive pathways 514, and position and number of the representations of the one or more sensors 516, 518.

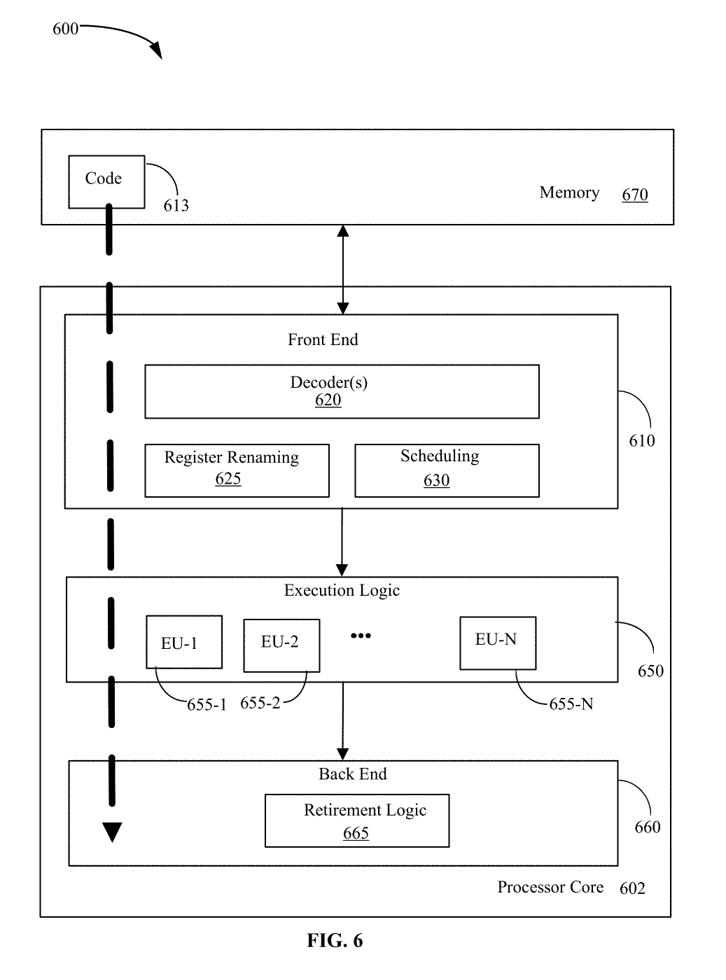

FIG. 6 is a block diagram 600 of a processor core 602 according to one embodiment. The processor core 602 may be the core for any type of processor, such as a micro-processor, an embedded processor, a digital signal processor (DSP), a network processor, or other device to execute code. Although only one processor core 602 is illustrated in FIG. 6, a processing element may alternatively include more than one of the processor core 602 illustrated in FIG. 6. The processor core 602 may be a single-threaded core or, for at least one embodiment, the processor core 602 may be multithreaded in that it may include more than one hardware thread context (or “logical processor”) per core.

FIG. 6 also illustrates a memory 670 coupled to the processor core 602. The memory 670 may be any of a wide variety of memories (including various layers of memory hierarchy) as are known or otherwise available to those of skill in the art. The memory 670 may include one or more code 613 instruction(s) to be executed by the processor core 602, wherein the code 613 may implement the method 300 (FIG. 3), already discussed. The processor core 602 follows a program sequence of instructions indicated by the code 613. Each instruction may enter a front end portion 610 and be processed by one or more decoders 620. The decoder 620 may generate as its output a micro operation such as a fixed width micro operation in a predefined format, or may generate other instructions, microinstructions, or control signals which reflect the original code instruction. The illustrated front end portion 610 also includes register renaming logic 625 and scheduling logic 630, which generally allocate resources and queue the operation corresponding to the convert instruction for execution.

The processor core 602 is shown including execution logic 650 having a set of execution units 655-1 through 655-N. Some embodiments may include a number of execution units dedicated to specific functions or sets of functions. Other embodiments may include only one execution unit or one execution unit that can perform a particular function. The illustrated execution logic 650 performs the operations specified by code instructions.

After completion of execution of the operations specified by the code instructions, back end logic 660 retires the instructions of the code 613. In one embodiment, the processor core 602 allows out of order execution but requires in order retirement of instructions. Retirement logic 665 may take a variety of forms as known to those of skill in the art (e.g., re-order buffers or the like). In this manner, the processor core 602 is transformed during execution of the code 613, at least in terms of the output generated by the decoder, the hardware registers and tables utilized by the register renaming logic 625, and any registers (not shown) modified by the execution logic 650.

Although not illustrated in FIG. 6, a processing element may include other elements on chip with the processor core 602. For example, a processing element may include memory control logic along with the processor core 602. The processing element may include I/O control logic and/or may include I/O control logic integrated with memory control logic. The processing element may also include one or more caches.

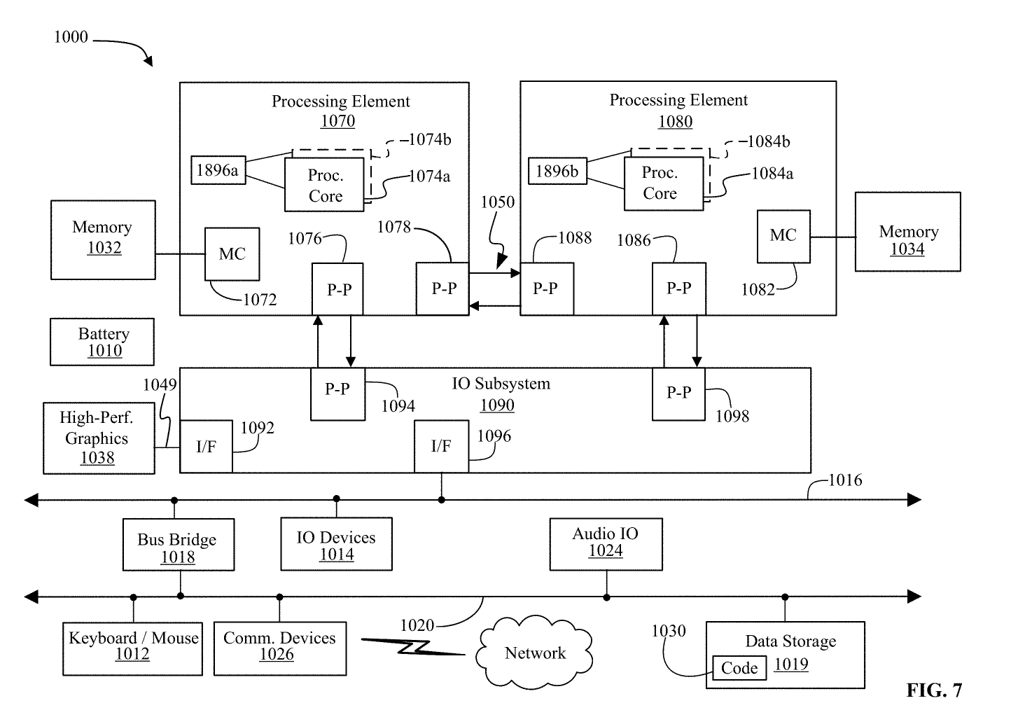

Referring now to FIG. 7, shown is a block diagram of a computing system 1000 embodiment in accordance with an embodiment. Shown in FIG. 7 is a multiprocessor system 1000 that includes a first processing element 1070 and a second processing element 1080. While two processing elements 1070 and 1080 are shown, it is to be understood that an embodiment of the system 1000 may also include only one such processing element.

The system 1000 is illustrated as a point-to-point interconnect system, wherein the first processing element 1070 and the second processing element 1080 are coupled via a point-to-point interconnect 1050. It should be understood that any or all of the interconnects illustrated in FIG. 7 may be implemented as a multi-drop bus rather than point-to-point interconnect.

As shown in FIG. 7, each of processing elements 1070 and 1080 may be multicore processors, including first and second processor cores (i.e., processor cores 1074a and 1074b and processor cores 1084a and 1084b). Such cores 1074a, 1074b, 1084a, 1084b may be configured to execute instruction code in a manner similar to that discussed above in connection with FIG. 6.

Each processing element 1070, 1080 may include at least one shared cache 1896a, 1896b. The shared cache 1896a, 1896b may store data (e.g., instructions) that are utilized by one or more components of the processor, such as the cores 1074a, 1074b and 1084a, 1084b, respectively. For example, the shared cache 1896a, 1896b may locally cache data stored in a memory 1032, 1034 for faster access by components of the processor. In one or more embodiments, the shared cache 1896a, 1896b may include one or more mid-level caches, such as level 2 (L2), level 3 (L3), level 4 (L4), or other levels of cache, a last level cache (LLC), and/or combinations thereof.

While shown with only two processing elements 1070, 1080, it is to be understood that the scope of the embodiments are not so limited. In other embodiments, one or more additional processing elements may be present in a given processor. Alternatively, one or more of processing elements 1070, 1080 may be an element other than a processor, such as an accelerator or a field programmable gate array. For example, additional processing element(s) may include additional processors(s) that are the same as a first processor 1070, additional processor(s) that are heterogeneous or asymmetric to processor a first processor 1070, accelerators (such as, e.g., graphics accelerators or digital signal processing (DSP) units), field programmable gate arrays, or any other processing element. There can be a variety of differences between the processing elements 1070, 1080 in terms of a spectrum of metrics of merit including architectural, micro architectural, thermal, power consumption characteristics, and the like. These differences may effectively manifest themselves as asymmetry and heterogeneity amongst the processing elements 1070, 1080. For at least one embodiment, the various processing elements 1070, 1080 may reside in the same die package.

The first processing element 1070 may further include memory controller logic (MC) 1072 and point-to-point (P-P) interfaces 1076 and 1078. Similarly, the second processing element 1080 may include a MC 1082 and P-P interfaces 1086 and 1088. As shown in FIG. 7, MC's 1072 and 1082 couple the processors to respective memories, namely a memory 1032 and a memory 1034, which may be portions of main memory locally attached to the respective processors. While the MC 1072 and 1082 is illustrated as integrated into the processing elements 1070, 1080, for alternative embodiments the MC logic may be discrete logic outside the processing elements 1070, 1080 rather than integrated therein.

The first processing element 1070 and the second processing element 1080 may be coupled to an I/O subsystem 1090 via P-P interconnects 1076 1086, respectively. As shown in FIG. 7, the I/O subsystem 1090 includes P-P interfaces 1094 and 1098. Furthermore, I/O subsystem 1090 includes an interface 1092 to couple I/O subsystem 1090 with a high performance graphics engine 1038. In one embodiment, bus 1049 may be used to couple the graphics engine 1038 to the I/O subsystem 1090. Alternately, a point-to-point interconnect may couple these components.

In turn, I/O subsystem 1090 may be coupled to a first bus 1016 via an interface 1096. In one embodiment, the first bus 1016 may be a Peripheral Component Interconnect (PCI) bus, or a bus such as a PCI Express bus or another third generation I/O interconnect bus, although the scope of the embodiments are not so limited.

As shown in FIG. 7, various I/O devices 1014 (e.g., speakers, cameras, sensors) may be coupled to the first bus 1016, along with a bus bridge 1018 which may couple the first bus 1016 to a second bus 1020. In one embodiment, the second bus 1020 may be a low pin count (LPC) bus. Various devices may be coupled to the second bus 1020 including, for example, a keyboard/mouse 1012, communication device(s) 1026, and a data storage unit 1019 such as a disk drive or other mass storage device which may include code 1030, in one embodiment. The illustrated code 1030 may implement the method 300 (FIG. 3), already discussed, and may be similar to the code 613 (FIG. 6), already discussed. Further, an audio I/O 1024 may be coupled to second bus 1020 and a battery 1010 may supply power to the computing system 1000.

Note that other embodiments are contemplated. For example, instead of the point-to-point architecture of FIG. 7, a system may implement a multi-drop bus or another such communication topology. Also, the elements of FIG. 7 may alternatively be partitioned using more or fewer integrated chips than shown in FIG. 7.

### ADDITIONAL NOTES AND EXAMPLES

Example 1 may include a garment enhancement apparatus comprising a material selector to select representations of one or more conductive materials and one or more decorative materials for a representation of a technology embedded garment, wherein the one or more conductive materials include conductive pathways, a sensor simulator to monitor at least one simulated biosignal at one or more representations of biometric sensors positioned along the conductive pathways of the one or more conductive materials, and a sensor positioner to position the representations of the one or more biometric sensors to reduce a number of the one or more representations of biometric sensors and increase an accuracy of the one or more representations of biometric sensors to measure the at least one simulated biosignal.

Example 2 may include the apparatus of Example 1, further comprising a design visualizer to edit the representation of the technology embedded garment and position the representations of the one or more biometric sensors.

Example 3 may include the apparatus of any one of Examples 1 to 2, wherein the sensor simulator is to measure a change in electric current produced by a sum of an electrical potential difference across one or more of a simulated tissue, organ, cell system or nervous system to monitor the at least one simulated biosignal, and measure a change in electric resistance produced by the conductive materials modified in a simulation to compare electrical properties between different simulated conductive materials including one or more of conductive threads, fabrics, inks or composites.

Example 4 may include the apparatus of Example 3, wherein the at least one simulated biosignal includes one or more of simulated bioelectrical signals, electrical signals, non-electrical signals or time-varying signals.

Example 5 may include the apparatus of Example 4, wherein the sensor positioner is to display a visual heat map and overlay that identifies positions and zones for the one or more representations of biometric sensors to monitor the at least one simulated biosignal.

Example 6 may include the apparatus of Example 5, wherein the sensor simulator is to determine positioning of the one or more representations of biometric sensors based on a physiological model of a wearer of the garment.

Example 7 may include the apparatus of Example 6, further comprises a three dimensional (3D) physiological avatar selector for selection of a 3D physiological avatar, wherein the 3D physiological avatar is to generate the at least one simulated biosignal for the 3D physiological avatar for input to the embedded technology in the garment, wherein the 3D physiological avatar is to be responsive to the embedded technology and the garment, and wherein the 3D physiological avatar is to be customized for one or more activities and one or more wearer profiles.

Example 8 may include the apparatus of Example 7, further comprises a technology embedded garment generator to generate a design pattern of the technology embedded garment in accordance with the design pattern.

Example 9 may include the apparatus of Example 7, wherein the technology embedded garment generator is to produce the technology embedded garment.

Example 10 may include a method of generating a technology embedded garment comprising selecting representations of one or more conductive materials and one or more decorative materials for a technology embedded garment, wherein the one or more conductive materials include conductive pathways, monitoring at least one simulated biosignal at one or more representations of biometric sensors positioned along the conductive pathways of the one or more conductive materials, and positioning the representations of the one or more biometric sensors to reduce a number of the one or more representations of biometric sensors and increase an accuracy of the one or more representations of biometric sensors to measure the at least one simulated biosignal.

Example 11 may include the Example 10, comprising visually presenting a design to edit the representation of the technology embedded garment and position the representations of the one or more biometric sensors.

Example 12 may include the method of any one of Examples 10 to 11, further comprising measuring a change in electric current produced by a sum of an electrical potential difference across one or more of a simulated tissue, organ, cell system or nervous system to monitor the at least one simulated biosignal, and measuring a change in electric resistance produced by the conductive materials modified in a simulation to compare electrical properties between different simulated conductive materials including one or more of conductive threads, fabrics, inks or composites.

Example 13 may include the method of Example 12, wherein the at least one simulated biosignal includes one or more of simulated bioelectrical signals, electrical signals, non-electrical signals or time-varying signals.

Example 14 may include the method of Example 13, further including displaying a visual heat map and overlay that identifies positions and zones for the one or more representations of biometric sensors to monitor the at least one simulated biosignal.

Example 15 may include the method of Example 14, further including determining a positioning of the one or more representations of biometric sensors based on a physiological model of a wearer of the garment.

Example 16 may include the method of Example 15, further comprising presenting a three dimensional (3D) physiological avatar selector for selection of a 3D physiological avatar selecting from an avatar selector, and generating, using the 3D physiological avatar, the at least one simulated biosignal for the 3D physiological avatar for input to the embedded technology in the garment, wherein the 3D physiological avatar is responsive to the embedded technology and the garment, and wherein the 3D physiological avatar is customized for one or more activities and one or more wearer profiles.

Example 17 may include the method of Example 16, further comprising generating a design pattern of the technology embedded garment, and producing the technology embedded garment in accordance with the design pattern.

Example 18 may include at least one computer readable storage medium comprising a set of instructions, which when executed by a computing device, cause the computing device to select representations of one or more conductive materials and one or more decorative materials for a technology embedded garment, wherein the one or more conductive materials include conductive pathways, monitor at least one simulated biosignal at one or more representations of biometric sensors positioned along the conductive pathways of the one or more conductive materials, and position the representations of the one or more biometric sensors to reduce a number of the one or more representations of biometric sensors and increase an accuracy of the one or more representations of biometric sensors to measure the at least one simulated biosignal.

Example 19 may include the at least one computer readable storage medium of Example 18, wherein the instructions, when executed, cause a computing device to visually present a design to edit the representation of the technology embedded garment and position the representations of the one or more biometric sensors.

Example 20 may include the at least one computer readable storage medium of any one of Examples 18 to 19, wherein the instructions, when executed, cause a computing device to measure a change in electric current produced by a sum of an electrical potential difference across one or more of a simulated tissue, organ, cell system or nervous system to monitor the at least one simulated biosignal, and measure a change in electric resistance produced by the conductive materials modified in a simulation to compare electrical properties between different simulated conductive materials including one or more of conductive threads, fabrics, inks or composites.

Example 21 may include the at least one computer readable storage medium of Example 20, wherein the at least one simulated biosignal is to include one or more of simulated bioelectrical signals, electrical signals, non-electrical signals or time-varying signals.

Example 22 may include the at least one computer readable storage medium of Example 21, wherein the sensor positioner is to display a visual heat map and overlay that identifies positions and zones for the one or more representations of biometric sensors to monitor the at least one simulated biosignal.

Example 23 may include the at least one computer readable storage medium of Example 22, wherein the instructions, when executed, cause a computing device to determine a positioning of the one or more representations of biometric sensors based on a physiological model of a wearer of the garment.

Example 24 may include the at least one computer readable storage medium of Example 23, wherein the instructions, when executed, cause a computing device to present a three dimensional (3D) physiological avatar selector for selection of a 3D physiological avatar, wherein the 3D physiological avatar is to generate the at least one simulated biosignal for the 3D physiological avatar for input to the embedded technology in the garment, wherein the 3D physiological avatar is to be responsive to the embedded technology and the garment, and wherein the 3D physiological avatar is to be customized for one or more activities and one or more wearer profiles.

Example 25 may include the at least one computer readable storage medium of Example 23, wherein the instructions, when executed, cause a computing device to generate a design pattern of the technology embedded garment and produce the technology embedded garment in accordance with the design pattern.

Example 26 may include a garment enhancement apparatus comprising: means for selecting representations of one or more conductive materials and one or more decorative materials for a technology embedded garment, wherein the one or more conductive materials is to include conductive pathways, means for monitoring at least one simulated biosignal at one or more representations of biometric sensors positioned along the conductive pathways of the one or more conductive materials, and means for positioning the representations of the one or more biometric sensors to reduce a number of the one or more representations of biometric sensors and increase an accuracy of the one or more representations of biometric sensors to measure the at least one simulated biosignal.

Example 27 may include the apparatus of Example 26, further comprising: means for visually presenting a design to edit the representation of the technology embedded garment and position the representations of the one or more biometric sensors, and means for measuring a change in electric current produced by a sum of an electrical potential difference across one or more of a simulated tissue, organ, cell system or nervous system to monitor the at least one simulated biosignal, wherein the at least one simulated biosignal is to include one or more of simulated bioelectrical signals, electrical signals, non-electrical signals or time-varying signals, and means for measuring a change in electric resistance produced by the conductive materials modified in a simulation to compare electrical properties between different simulated conductive materials including one or more of conductive threads, fabrics, inks or composites.

Example 28 may include the apparatus of any one of Examples 26 to 27, further including: means for displaying a visual heat map and overlay to identify positions and zones for the one or more representations of biometric sensors to monitor the at least one simulated biosignal, and means for determining a positioning of the one or more representations of biometric sensors based on a physiological model of a wearer of the garment.

Example 29 may include the apparatus of Example 28, means for presenting a three dimensional (3D) physiological avatar selector for selection of a 3D physiological avatar selecting from an avatar selector, and means for generating, using the 3D physiological avatar, the at least one simulated biosignal for the 3D physiological avatar for input to the embedded technology in the garment, wherein the 3D physiological avatar is to responsive to the embedded technology and the garment, and wherein the 3D physiological avatar is to customized for one or more activities and one or more wearer profiles.

Example 30 may include the apparatus of Example 29, further comprising: means for generating a design pattern of the technology embedded garment, and means for producing the technology embedded garment in accordance with the design pattern. Embodiments are applicable for use with all types of semiconductor integrated circuit (“IC”) chips. Examples of these IC chips include but are not limited to processors, controllers, chipset components, programmable logic arrays (PLAs), memory chips, network chips, systems on chip (SoCs), SSD/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the computing system within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections. In addition, the terms “first”, “second”, etc. may be used herein only to facilitate discussion, and carry no particular temporal or chronological significance unless otherwise indicated.

As used in this application and in the claims, a list of items joined by the term “one or more of” may mean any combination of the listed terms. For example, the phrases “one or more of A, B or C” may mean A; B; C; A and B; A and C; B and C; or A, B and C.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

We claim:

1. An apparatus comprising:
a material selector to select representations of one or more conductive materials and one or more decorative materials for a representation of a technology embedded garment, wherein the one or more conductive materials include conductive pathways,
a sensor simulator to monitor at least one simulated biosignal at one or more representations of biometric sensors positioned along the conductive pathways of the one or more conductive materials, and
a sensor positioner to position the representations of the one or more biometric sensors to reduce a number of the one or more representations of biometric sensors and increase an accuracy of the one or more representations of biometric sensors to measure the at least one simulated biosignal.

2. The apparatus of claim 1, further comprising a design visualizer to edit the representation of the technology embedded garment and position the representations of the one or more biometric sensors.

3. The apparatus of claim 1, wherein the sensor simulator is to:
measure a change in electric current produced by a sum of an electrical potential difference across one or more of a simulated tissue, organ, cell system or nervous system to monitor the at least one simulated biosignal; and
measure a change in electric resistance produced by the conductive materials modified in a simulation to compare electrical properties between different simulated conductive materials including one or more of conductive threads, fabrics, inks or composites.

4. The apparatus of claim 3, wherein the at least one simulated biosignal includes one or more of simulated bioelectrical signals, electrical signals, non-electrical signals or time-varying signals.

5. The apparatus of claim 1, wherein the sensor positioner is to display a visual heat map and overlay that identifies positions and zones for the one or more representations of biometric sensors to monitor the at least one simulated biosignal.

6. The apparatus of claim 1, wherein the sensor simulator is to determine positioning of the one or more representations of biometric sensors based on a physiological model of a wearer of the garment.

7. The apparatus of claim 1, further comprises a three dimensional (3D) physiological avatar selector for selection of a 3D physiological avatar, wherein the 3D physiological avatar is to generate the at least one simulated biosignal for the 3D physiological avatar for input to the embedded technology in the garment, wherein the 3D physiological avatar is to be responsive to the embedded technology and the garment, and wherein the 3D physiological avatar is to be customized for one or more activities and one or more wearer profiles.

8. The apparatus of claim 1, further comprises a technology embedded garment generator to generate a design pattern of the technology embedded garment in accordance with the design pattern.

9. The apparatus of claim 8, wherein the technology embedded garment generator is to produce the technology embedded garment.

10. A method comprising:
selecting representations of one or more conductive materials and one or more decorative materials for a technology embedded garment, wherein the one or more conductive materials include conductive pathways,
monitoring at least one simulated biosignal at one or more representations of biometric sensors positioned along the conductive pathways of the one or more conductive materials, and
positioning the representations of the one or more biometric sensors to reduce a number of the one or more representations of biometric sensors and increase an accuracy of the one or more representations of biometric sensors to measure the at least one simulated biosignal.

11. The method of claim 10, comprising visually presenting a design to edit the representation of the technology embedded garment and position the representations of the one or more biometric sensors.

12. The method of claim 10, further comprising:
measuring a change in electric current produced by a sum of an electrical potential difference across one or more of a simulated tissue, organ, cell system or nervous system to monitor the at least one simulated biosignal; and
measuring a change in electric resistance produced by the conductive materials modified in a simulation to compare electrical properties between different simulated conductive materials including one or more of conductive threads, fabrics, inks or composites.

13. The method of claim 12, wherein the at least one simulated biosignal includes one or more of simulated bioelectrical signals, electrical signals, non-electrical signals or time-varying signals.

14. The method of claim 10, further including displaying a visual heat map and overlay that identifies positions and zones for the one or more representations of biometric sensors to monitor the at least one simulated biosignal.

15. The method of claim 10, further including determining a positioning of the one or more representations of biometric sensors based on a physiological model of a wearer of the garment.

16. The method of claim 15, further comprising:
presenting a three dimensional (3D) physiological avatar selector for selection of a 3D physiological avatar selecting from an avatar selector; and
generating, using the 3D physiological avatar, the at least one simulated biosignal for the 3D physiological avatar for input to the embedded technology in the garment, wherein the 3D physiological avatar is responsive to the embedded technology and the garment, and wherein the 3D physiological avatar is customized for one or more activities and one or more wearer profiles.

17. The method of claim 10, further comprising:
generating a design pattern of the technology embedded garment; and
producing the technology embedded garment in accordance with the design pattern.

18. At least one computer readable storage medium comprising a set of instructions, which when executed by a computing device, cause the computing device to:
select representations of one or more conductive materials and one or more decorative materials for a technology embedded garment, wherein the one or more conductive materials include conductive pathways,
monitor at least one simulated biosignal at one or more representations of biometric sensors positioned along the conductive pathways of the one or more conductive materials, and
position the representations of the one or more biometric sensors to reduce a number of the one or more representations of biometric sensors and increase an accuracy of the one or more representations of biometric sensors to measure the at least one simulated biosignal.

19. The at least one computer readable storage medium of claim 18, wherein the instructions, when executed, cause a computing device to visually present a design to edit the representation of the technology embedded garment and position the representations of the one or more biometric sensors.

20. The at least one computer readable storage medium of claim 18, wherein the instructions, when executed, cause a computing device to:
measure a change in electric current produced by a sum of an electrical potential difference across one or more of a simulated tissue, organ, cell system or nervous system to monitor the at least one simulated biosignal; and
measure a change in electric resistance produced by the conductive materials modified in a simulation to compare electrical properties between different simulated conductive materials including one or more of conductive threads, fabrics, inks or composites.

21. The at least one computer readable storage medium of claim 20, wherein the at least one simulated biosignal is to include one or more of simulated bioelectrical signals, electrical signals, non-electrical signals or time-varying signals.

22. The at least one computer readable storage medium of claim 18, wherein the sensor positioner is to display a visual heat map and overlay that identifies positions and zones for the one or more representations of biometric sensors to monitor the at least one simulated biosignal.

23. The at least one computer readable storage medium of claim 18, wherein the instructions, when executed, cause a computing device to determine a positioning of the one or more representations of biometric sensors based on a physiological model of a wearer of the garment.

24. The at least one computer readable storage medium of claim 23, wherein the instructions, when executed, cause a computing device to present a three dimensional (3D) physiological avatar selector for selection of a 3D physiological avatar, wherein the 3D physiological avatar is to generate the at least one simulated biosignal for the 3D physiological avatar for input to the embedded technology in the garment, wherein the 3D physiological avatar is to be responsive to the embedded technology and the garment, and wherein the 3D physiological avatar is to be customized for one or more activities and one or more wearer profiles.

25. The at least one computer readable storage medium of claim 18, wherein the instructions, when executed, cause a computing device to:
generate a design pattern of the technology embedded garment; and
produce the technology embedded garment in accordance with the design pattern.
